(19) United States
(12) Patent Application Publication
(10) Pub. No.: US 2022/0331152 A1
(43) Pub. Date: Oct. 20, 2022

(54) PROVIDING THERAPEUTIC RECOVERY USING HEAT DISSIPATION FOR WEARABLE APPLICATIONS

(71) Applicant: Cube Recovery Company, Stamford, CT (US)

(72) Inventors: Aaron Young, Brooklyn, NY (US); Steve Lepke, Wakefield, MA (US); Vincent Lambert, Salisbury, MA (US); John Li, Brooklyn, NY (US)

(21) Appl. No.: 17/725,229

(22) Filed: Apr. 20, 2022

Related U.S. Application Data
(60) Provisional application No. 63/177,284, filed on Apr. 20, 2021.
(60) Provisional application No. 63/244,073, filed on Sep. 14, 2021.
(60) Provisional application No. 63/310,046, filed on Feb. 14, 2022.

(51) Int. Cl. A61F 7/00

(52) U.S. Cl. CPC A61F 7/007; A61F 2007/0058; A61F 2007/0078

(57) ABSTRACT

A wearable device includes a housing to encompass a portion of a body part, biometric sensors coupled to the housing, a controller coupled to the housing and biometric sensors, a communication channel for communicating with external devices and a temperature regulator to controllably adjust a temperature of the housing. The wearable device is configured to provide remote controlled heating and cooling of the body part. The wearable device may include an evaporative heat exchanger and a thermoelectric heat pump. The wearable device may provide means for dissipating heat through vaporization of a substance or using an advective flux and phase transition.

### CROSS-REFERENCE TO RELATED APPLICATIONS

The present application claims the benefit of U.S. Prov. Pat. App. 63/177,284, filed 20 Apr. 2021, titled DEVICE FOR AND METHOD OF THERAPEUTIC ICING AND HEATING; U.S. Prov. Pat. App 63/244,073, filed 14 Sep. 2021, titled HEAT DISSIPATION SYSTEM FOR WEARABLE APPLICATIONS; and U.S. Prov. Pat. App. 63/310,046, titled Heat Dissipation System for Wearable Applications, filed 14 Feb. 2022. The entire content of each afore-listed earlier-filed application is hereby incorporated by reference for all purposes.

### BACKGROUND

The present disclosure relates generally to a therapeutic system including a heat dissipation system that provides a mechanism for heating and cooling to be used in wearable device applications.

The feasibility of wearable technology is often dependent on weight and volume in some use cases. Users generally prefer lighter-weight wearable devices, which can be challenging for use cases involving heat transfer, where heavy, bulky components are often used.

### SUMMARY

Millions of Americans suffer from chronic injuries every year that require therapeutic icing. A cryo-pod, an ice bath, and an ice bag have been used to recover from a chronic injury.

Shortcomings of conventional methods of recovering from a chronic injury include non-portability of a cryo-pod, waste of water in an ice bath, single usage of an ice bag, toxic chemicals in an ice bag, and assistance that may be required with an ice bag.

Thus, there is a need for an icing and heating device that is portable, does not waste water, does not use toxic chemicals, is not usable only once, and does not require an assistant to be physically present.

The following is a non-exhaustive listing of some aspects of the present techniques. These and other aspects are described in the following disclosure.

Some aspects include a heat dissipation system for wearable devices that uses vaporization of a harmless substance, in some cases, combined with advection.

### BRIEF DESCRIPTION OF THE DRAWINGS

The above-mentioned aspects and other aspects of the present techniques will be better understood when the present application is read in view of the following figures in which like numbers indicate similar or identical elements:

FIG. 1A is a first perspective view of a wearable device for a knee of a wearer shown in accordance with some embodiments.

FIG. 1B is a second perspective view of a wearable device for a knee of a wearer shown in accordance with some embodiments.

FIG. 1C is a third perspective view of a wearable device for a knee of a wearer shown in accordance with some embodiments.

FIG. 1D is a plan view of a Peltier element in accordance with some embodiments.

FIG. 1E is a block diagram that illustrates energy flow through a wearable device in accordance with some embodiments.

FIG. 1F illustrates a schematic of a wearable device and communication devices in accordance with some embodiments.

FIG. 2 is an illustration of a wearable device for a knee or an elbow and a mobile device in accordance with some embodiments.

FIG. 3 is an illustration of a wearable device for a foot and the mobile device in accordance with some embodiments.

FIG. 4 is an illustration of a wearable device for a chest or a back and the mobile device in accordance with some embodiments.

FIG. 5 is an illustration of a wearable device for a waist or a groin and the mobile device in accordance with some embodiments.

FIG. 6 is an illustration of a wearable device for a hand and the mobile device in accordance with some embodiments.

FIG. 7 is an illustration of a wearable device for a head or a forehead and the mobile device in accordance with some embodiments.

FIG. 8 is an illustration of a wearable device for an eye, a nose, an ear, or a mouth and the mobile device in accordance with some embodiments.

FIG. 9 is a method of icing and heating using a wearable device in accordance with some embodiments.

FIG. 10 is a block diagram that illustrates energy flow in accordance with some embodiments.

FIG. 11 is a schematic that illustrates energy flow in accordance with some embodiments.

FIG. 12 illustrates a block diagram of a representative system including one or more wearable devices in accordance with some embodiments.

FIG. 13 illustrates an example computing device by which the above techniques may be implemented.

While the present techniques are susceptible to various modifications and alternative forms, specific embodiments thereof are shown by way of example in the drawings and will herein be described in detail. The drawings may not be to scale. It should be understood, however, that the drawings and detailed description thereto are not intended to limit the present techniques to the particular form disclosed, but to the contrary, the intention is to cover all modifications, equivalents, and alternatives falling within the spirit and scope of the present techniques as defined by the appended claims.

### DETAILED DESCRIPTION

To mitigate the problems described herein, the inventors had to both invent solutions and, in some cases just as importantly, recognize problems overlooked (or not yet foreseen) by others in the field of heat transfer and medical device design. Indeed, the inventors wish to emphasize the difficulty of recognizing those problems that are nascent and will become much more apparent in the future should trends in industry continue as the inventors expect. Further, because multiple problems are addressed, it should be understood that some embodiments are problem-specific, and not all embodiments address every problem with traditional systems described herein or provide every benefit described herein. That said, improvements that solve various permutations of these problems are described below.

For cooling applications involving wearable devices, Peltier thermoelectric heat pumps (referred to as “Peltier devices” or “Peltier modules” interchangeably) may be used for cooling body parts of the user. Peltier devices transport heat using energy provided by electrical current flow. However, the efficiency of Peltier cooling is highly dependent on the temperature difference across the module. When a large temperature difference is built up across the module, the temperature gradient drives heat flux in the reverse direction as the direction that the heat is intended to be transported, and the coefficient of performance is significantly decreased. These cases are problematic due to the unfavorable scaling relation regarding the resistive heating of the Peltier module itself combined with increased heat backflow as compared to the heat transported per unit current, especially since larger current leads to increased resistive heating of the module itself, which in turn, requires more heat in total to be dissipated. Therefore, it is beneficial in some cases to remove heat from the Peltier module (e.g., from a hot side of the module) to keep the temperature of the thermal transport energy destination low. In some embodiments, the wearable device cools the module solely with air cooling by ambient, untreated air. However, due to the low thermal conductivity and volumetric heat capacity of air, the cooling provided tends to be insufficient for transporting large quantities of heat at a high rate in some use cases (which is not to suggest that this approach or any other is disclaimed). Some embodiments enhance transfer of thermal energy away from the side of the Peltier module (e.g., expel thermal energy) where heat is being deposited through dissipating thermal energy to the surrounding environment.

Icing is a common recommended therapy for reducing inflammation and pain during exercise. Since the body is mostly comprised of water, cooling is energetically expensive given that water has a relatively high heat capacity. Ice packs depend on the enthalpy of fusion of ice to absorb the thermal energy. For wearable electronic devices to provide similar or equivalent cooling as compared to a passive system like an ice pack, a comparable amount of energy that is absorbed by the enthalpy of fusion of ice may be transported away from the body (in some cases, during a comparable amount of time). During an icing session, around ˜300 grams of ice may melt over a time period of less than one hour (like less than 30 minutes), leading to the removal of ˜1×10\hat{ }5 J of energy from the body within 600-1200 seconds. Assuming a reasonable coefficient of performance of 1 for the heat pump (like a Peltier thermoelectric heat pump), ˜2×10\hat{ }5 J may be ejected into the environment to provide a comparable amount of cooling as an ice pack, given that the heat dissipated may include the energy used by the heat pump to transport thermal energy away from the body. The amount of heat to be removed from the system is, in some cases, approximately equivalent to the amount of energy required to vaporize on the order of 100 grams of liquid water, which has a latent heat of vaporization nearly an order of magnitude larger than its latent heat of fusion.

Some embodiments offset the large quantity of energy to be dissipated through the use of vaporization of a substance (which may be carried away from the heat pump by advection or through evaporation at the site of the heat pump or heat sink thermally coupled thereto), which may include any or a combination of the following compounds or mixtures: water, propylene glycol, glycerin, or any other harmless substance that has a significantly large latent heat of vaporization greater than 15000 J/mol or 300 J/g, such as water, with a latent heat of vaporization of 2257 J/g at ambient pressure at sea level. The aforementioned vaporization process leverages the latent heat of vaporization of the substance and can remove more heat per mass and volume than some other approaches, such as those relying on latent heat of fusion (which is not to suggest that such approaches are disclaimed, as both approaches could be combined in some embodiments). Additionally, air may be transported through the device for the purpose of carrying the vapor from vaporization of the working fluid by the device to the surrounding environment, wherein thermal energy is removed through a combination of (advection driven) convective cooling and evaporative cooling. The advection may be provided by a fan, blower, pump, motor, compressor, or any other system that facilitates bulk transport of gas, air, and/or other fluid, for the purpose of removing the vapor and carrier gas (like ambient air) that has absorbed heat from the device. The energy balance is expected to lead to a greater capability to cool given the limited quantity of energy that is available to power wearable devices, especially due to the lack of need to recondense the harmless substance that is being vaporized and ejected into the surrounding environment external to the thermodynamic system of interest.

In some embodiments, the wearable device includes a heat dissipation system that uses both an advective flux coupled to heat transfer and a phase transition to carry thermal energy away from the heat sink. In some cases, this system may be integrated into the wearable devices described in the documents incorporated by reference. The system may use the vaporization of a harmless substance (like water) to absorb heat at the sites where thermal energy from the body and/or from resistive heating of electronic components are transported to, and uses advection to carry the vaporized substance away.

Some embodiments function with a variety of control systems (examples of which are described in the documents incorporated by reference), such as intermittent or alternating power of vapor according to time intervals or measurement of temperature, that is vapor controlled by a thermo-sensor activated or deactivated at specified temperatures; including vapor control of system, such as deactivating the device power when vapor level is empty, and/or generating a display message to “fill or refill” vapor source, for example. Some embodiments may operate at less than 24 volts, for example less than or equal to 12 volts, to mitigate weight, noise, and thermal load from higher-voltage components.

In some embodiments, a wearable device for providing therapeutic recovery can be coupled with a profile that is set by a person (e.g., a user/wearer of an embodiment, a trainer of the user, etc.) with a recovery program for a user (e.g., an athlete), where the athlete inputs progress (e.g., temperature, time, diet, sleep, stress level, etc.). For example, an athlete has a knee injury, and his trainer wants him to adhere to a recovery program that includes repeated icing and heating of the knee. The athlete can utilize the wearable device contemplated herein that includes an integrated communication device that allows the trainer to control/modify/oversee the recovery of the athlete's knee. In use, the athlete will apply the wearable device to his knee and turn the wearable device on. The trainer may then remotely configure a 20-minute recovery program that includes the wearable device applying heat for 5 minutes, cooling for 5 minutes, and so on until the 20 minutes is up. Alternatively, the athlete may utilize an onboard display to manually configure the wearable device for example, if the wearable device is too hot or cold for his comfort.

In some embodiments, a wearable device for providing therapeutic recovery is wearable on one or more portions of a wearer's body and provides icing or heating in intervals. For example, a user may program an embodiment to provide heat and/or cold (e.g., icing) in user-definable time intervals (e.g., 15 minutes, 20 minutes). Some embodiments may also be operated manually through a device interface or an application or app (e.g., a Bluetooth™ application).

In some embodiments, the wearable device for providing therapeutic recovery securely transmits a message to another person (e.g., a doctor, a physical therapist, a trainer) and securely receives messages from the same. The message may concern an instruction from the other person to the user of some embodiments, progress of the user, and another type of communication between the other person and the user.

In some embodiments, a wearable device for providing therapeutic recovery may be used on various body parts (e.g., an ankle, a knee, a back, a wrist, a hip, a shoulder). Some embodiments may be in the form of a vest. Additionally, the wearable device may be configured for use on multiple body parts (e.g., both a wrist and an ankle).

In some embodiments, a wearable device for providing therapeutic recovery may be used by various people (e.g., a high school athlete, a college athlete, a professional athlete, a person recovering from surgery, a laborer). In some embodiments, a wearable device for providing therapeutic recovery may also be used by a person (e.g., a first responder) on another person (e.g., an injured person). The wearable device may be used for medical purposes (e.g., medical icing) and for enhancing athletic performance (e.g., performance cooling). Medical purposes include providing icing or heating concerning hip replacement surgery, knee replacement surgery, anterior cruciate ligament (ACL) surgery, medial collateral ligament (MCL) surgery, rotator cup surgery, herniated disc surgery, back pain, wrist pain, hand pain, ankle injury, etc.

In some embodiments, the wearable device for providing therapeutic recovery is portable, powered by a rechargeable battery, provides cold therapy, is wearable, provides heating and/or cooling in user-definable intervals, and is operable via an application.

Expected advantages of various embodiments described herein include the various forms of improvement to thermal efficiency described, temperature control (e.g., adjustability), time control (e.g., adjustability), communication capability (e.g., data updates for efficient assessment), wearability (e.g., long lasting durable mobility), sustainability (e.g., eliminates single-use and reduces water waste), and operability via an application (e.g., allows access via a mobile device). But embodiments are not limited to systems affording these advantages, as various approaches are described that can be used independent and some address other problems, which is not to suggest that any other description is limiting. It is understood that the terms “hotter” and “cooler” refer to temperatures higher and/or lower than a current temperature of the discussed object.

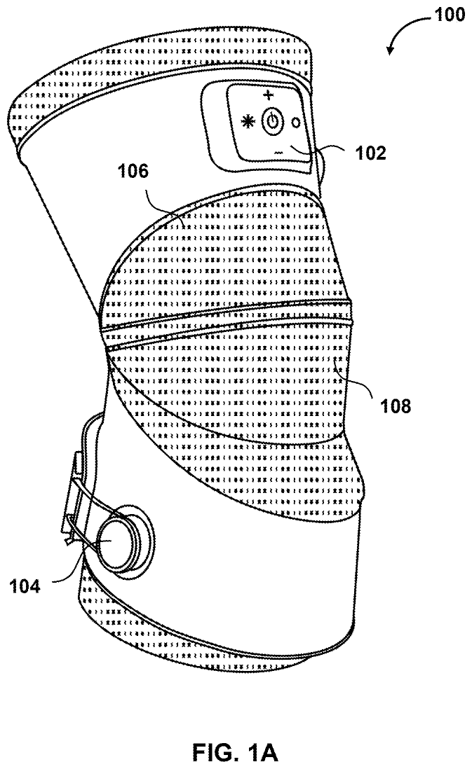

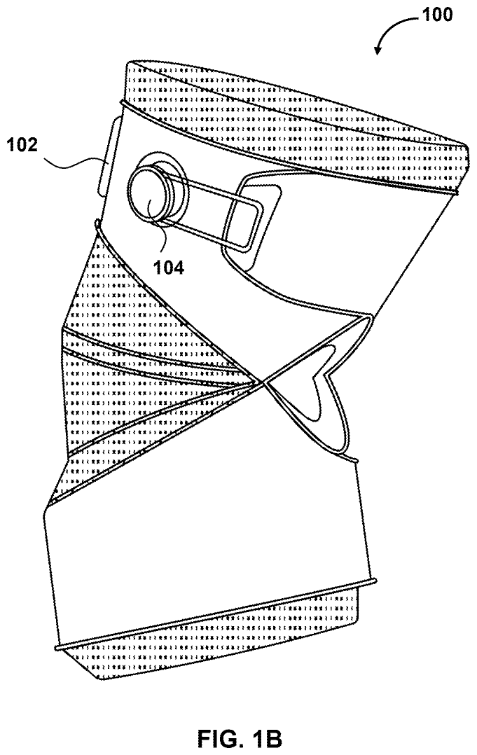

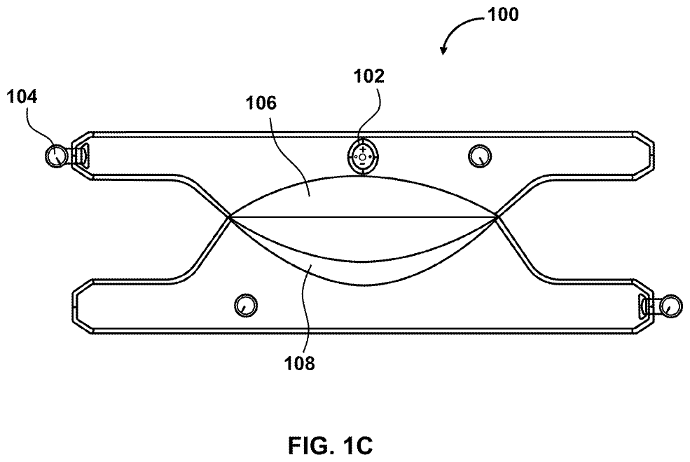

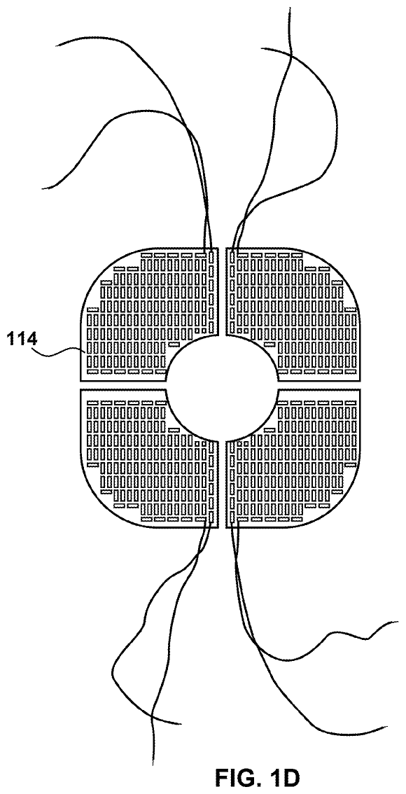

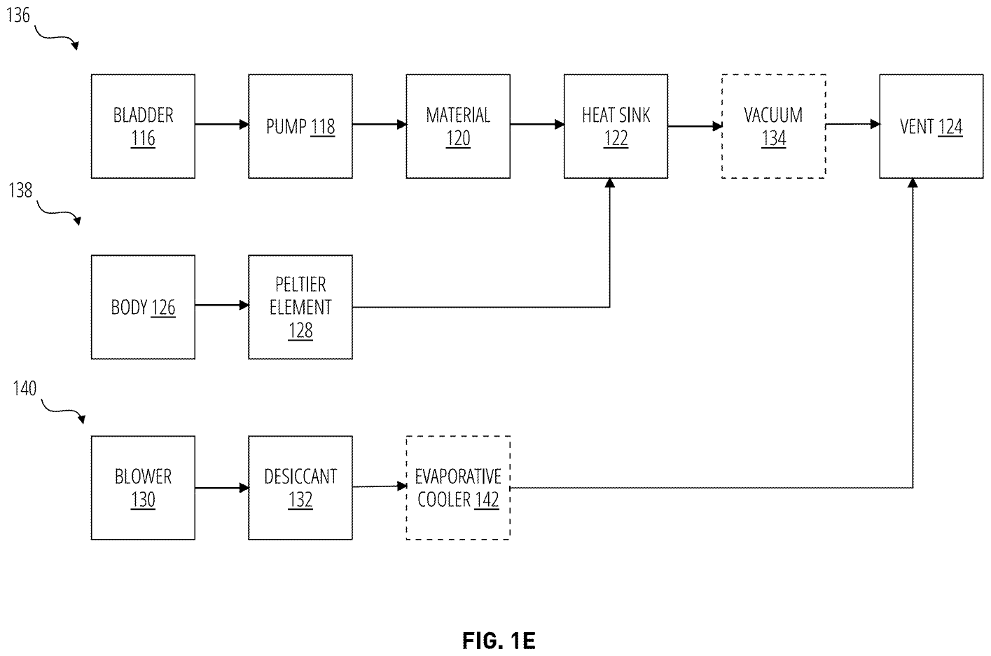

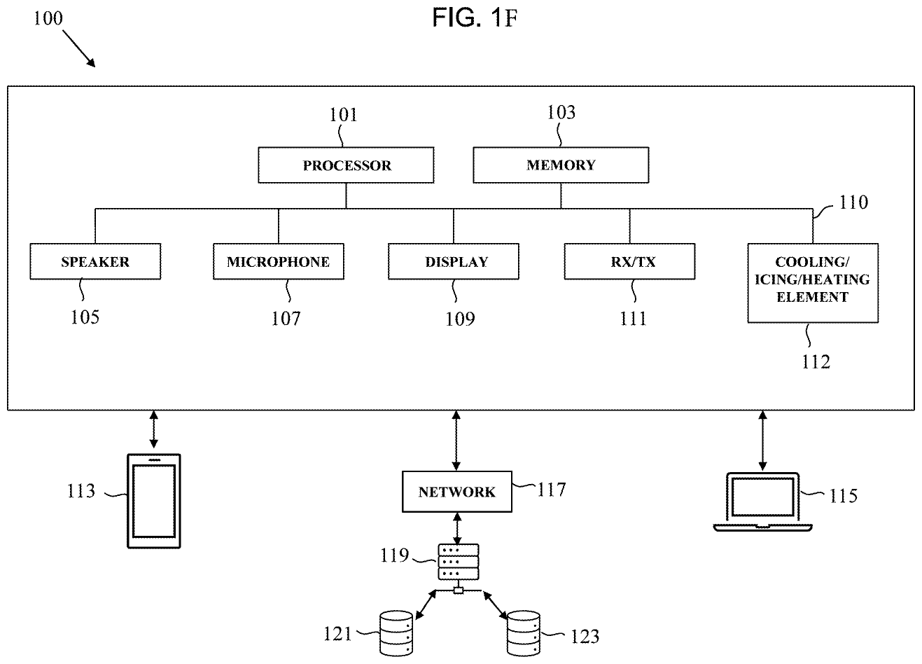

FIG. 1A is a first perspective view of a wearable device for a knee of a wearer shown in accordance with some embodiments. Wearable device 100 is configured to be worn on or around a knee and, in some embodiments, has a flexible structure that allows the user to remain ambulatory when wearing the device. In some embodiments, wearable device 100 is communicably coupled with a mobile device (e.g., mobile device 113, FIG. 1E). As shown in FIG. 1A, a front view of wearable device 100 includes a control panel 102, adjustment control 104, and flexible regions 106 and 108.

Some embodiments may include elastomeric sleeve or other wearable mounting platform (like with laces, Velcro™, or straps) configured to place an active surface of the cooling units (which may also be heating units) adjacent the skin of a human wearing the device. Examples include the sleeves described in the documents incorporated by reference above, such as a sleeve configured to fit over a user's joint, like their knee or elbow. Some embodiments may include a plurality of cooling/heating units arrayed around a strap or sleeve (or other article of clothing, like a hat, gloves, shoes, a vest, a coat, shorts, underwear, pants, socks, wristbands, headbands, waistbands, etc.). In some cases, each cooling unit may be independently controllable via a microcontroller of the apparatus, and in some cases, sub-regions of the cooling unit may be independently addressable to vary cooling or heating. Some embodiments may include a plurality of such cooling units, like two, four, six, eight, or more.

FIG. 1B is a second perspective view of a wearable device for a knee of a wearer shown in accordance with some embodiments. FIG. 1B shows a rear-perspective view of wearable device 100 that includes a control panel 102, adjustment control 104, and flexible regions 106 and 108.

FIG. 1C is a plan view of an un-rolled wearable device (like that of FIGS. 1A-B) for a knee of a wearer shown in accordance with some embodiments. FIG. 1C shows a top plan view of the wearable device including control panel 102, adjustment control 104, and flexible regions 106 and 108. It is understood that various other adjustment controls, flexible regions, and features described herein may not be shown or labeled in FIGS. 1A-1C but may be included. In some embodiments, the features described with reference to subsequent figures are present in the design of FIGS. 1A-C, as is the case with the other examples of depicted wearable devices.

FIG. 1D is a plan view of a flexible Peltier element in accordance with some embodiments. The device may be made from material available from TEGway, at #202, 56 Sinilseo-ro 67beon-gil, Daedeok-gu, Daejeon, Republic of Korea (34325). Wearable device 100 may include one or more Peltier elements (e.g., Peltier thermoelectric heat pumps, Peltier devices, Peltier modules) described herein. In some embodiments, Peltier element 114 is a flat and flexible construction suitable for installation in wearable devices. As shown in FIG. 1D, Peltier element 114 has a substantially flat shape and surface arranged in a configuration that provides an aperture in the middle of the element. The shape and configuration of Peltier element 114 is to allow the Peltier element to flexibly form around a knee of a wearer and the aperture to provide space for a kneecap. Various other configurations of a flexible Peltier element are contemplated herein with regards to wearable devices. In some embodiments, the device 114 may be embedded in the wearable devices described herein.

FIG. 1E is a block diagram that illustrates energy and mass flow through a wearable device in accordance with some embodiments. Additional features (like interfaces) present in some embodiments are described below. FIG. 1E includes a path of a working fluid 136, a path of heat 138 (the direction of which may be reversed in some cases), and a path of air 140 through or at wearable device (e.g., wearable device 100) in accordance with some embodiments.

In some embodiments, the path of working fluid 136 begins at a bladder 116 configured to store, hold, maintain working fluid (e.g., water) coupled to a pump 118 configured to circulate the working fluid to material 120 (e.g., housing of the wearable device 100), placing the working fluid in thermal contact with a heat sink 122 (e.g., an absorptive material or ramified channels that accept thermal energy) to an exhaust vent 124. In some embodiments blower 130 is coupled to heat sink 122 (not shown in diagram of FIG. 1E) to assist in dissipating heat from the heat sink. In some embodiments, the working fluid is transmitted from vent 124 as the working fluid evaporates with the help of an ultrasonic transducer. In some embodiments, a vacuum 134 is configured downstream of the heat sink in the path of the working fluid 136 to draw the working fluid from the bladder 116 to material 120 and heat sink 122. In some embodiments, the path of the working fluid includes both the pump 118 and vacuum 134. In some embodiments, the path of the working fluid includes either the pump 118 or vacuum 134.

The path of heat 138 may begin at a body 126 (e.g., body or body part of a wearer of the wearable device). Heat from the body 126 is transferred to Peltier element 128 and then to heat sink 122. In some embodiments, heat sink 122 includes multiple heat sinks for each path (e.g., path of working fluid 136 and path of heat 138). In some embodiments, both the path of heat 138 and path of working fluid 136 use the same heat sink 122.

The path of air 140 may include a blower 130 (e.g., a fan) to blow gas (e.g., air) across desiccant 132 to cool the gas and surrounding space. In some embodiments, the air passed across desiccant 132 is cooled air at a lower temperature than the air in a surrounding space (e.g., within 10 cubic centimeters) and the cooled air is directed to vent 124 having one or more exhaust ports. In some embodiments, the air passed across desiccant 132 is passed through evaporative cooler 142. In some embodiments, evaporative cooler 142 is coupled to one or more modules of working fluid path 136 to use a working fluid (e.g., water) to evaporate the working fluid and subsequently vent the air and evaporated working fluid through vent 124. For example, some embodiments may generate a mist of water with the ultrasonic transducer, spray nozzle, atomizer, or the like.

In some embodiments, one or more of the paths 136-140 are used to provide heating and cooling of the wearable device. It is understood that any of the modules used in the paths 136-140 may be used interchangeably or in various configurations not shown in FIG. 1E. The configuration of modules in FIG. 1E is not intended to be limiting but rather provide an example configuration.

FIG. 1F is a schematic of a wearable device 100 and communication devices of the some embodiments.

Referring to FIG. 1F, the wearable device 100 includes a processor 101, a memory 103, a speaker 105, a microphone 107, a display 109, a transceiver (RX/TX) 111, and an icing/heating element 112. In some embodiments, the icing and heating elements are discrete elements that functionally operate together. In other words, the icing element is a standalone element as well as the heating element being a standalone element. Both the icing and heating element may be configured to operate independently or concurrently. The icing and heating elements may be used and referred to herein as a temperature regulator where the icing and heating elements work together to provide an increase and/or decrease in temperature of the wearable device. In some embodiments, the temperature of the icing/heating element 112 is configured to fluctuate between a hot temperature (providing heat) and a cold temperature (providing cooling).

The processor 101, the memory 103, the speaker 105, the microphone 107, the display 109, the RX/TX 111, and the icing/heating element 112 are coupled to each other via a bidirectional bus 110.

In some embodiments, the processor 101 is configured to control the icing/heating element 112, the memory 103, the speaker 105, the microphone 107, the display 109, the RX/TX 111, and the icing/heating element 112 via the bidirectional bus 110.

The memory 103 stores instructions for controlling the icing/heating element 112 (e.g., temperature regulator), a user's icing/heating profile, which instruct the processor 101 on how to control the speaker 105, the microphone 107, the display 109, the RX/TX 111, and the icing/heating element 112. The processor 101 also stores in the memory 103 results of operating the icing/heating element 112, instructions input manually by the user via the display 109, and instructions received via the RX/TX 111.

The speaker 105 outputs audio from the processor 101 concerning operation of the wearable device 100 and may provide outputs of audible messages received via the RX/TX 111.

The microphone 107 inputs audio from the user when the user communicates with another party via the RX/TX 111 or when the user provides audio input to the wearable device 100. For example, an athlete is using the wearable device and the wearable device is communicably coupled with the trainer's cell phone. The trainer may monitor the athlete's progress using the wearable device. In some embodiments, the trainer may send one or more instructions to the wearable to adjust a temperature or adjust a timing of the provided therapy session. In some embodiments, a remote device (e.g., trainer's cell phone) can provide emergency notifications that shut down the wearable device.

The display 109 may comprise a touch screen with provides visual output to the user on all aspects of the operation of the wearable device, results of the operation of the wearable device 100, and an input capability to allow the user to manually input instructions and messages into the wearable device 100. The display 109 may additionally and/or alternatively include mechanical buttons or switches to control the wearable device.

The RX/TX 111 may be configured to send and receive messages between the user of the wearable device and another person or electronic device (e.g., a doctor, a trainer, a communication device of the user, a communication device of another party). The communication devices of the user and another party include a mobile device 113, a laptop computer 115, a server 119, and computers 121 and 123 connected to the server 119. Messages to and from the RX/TX 111 may go directly to the communication device (e.g., via Bluetooth communication) or via a network 117 (e.g., the Internet).

The icing/heating element 112 includes icing elements and heating elements that provide icing and heating, respectively, in accordance with the instructions and user profile stored in the memory 103.

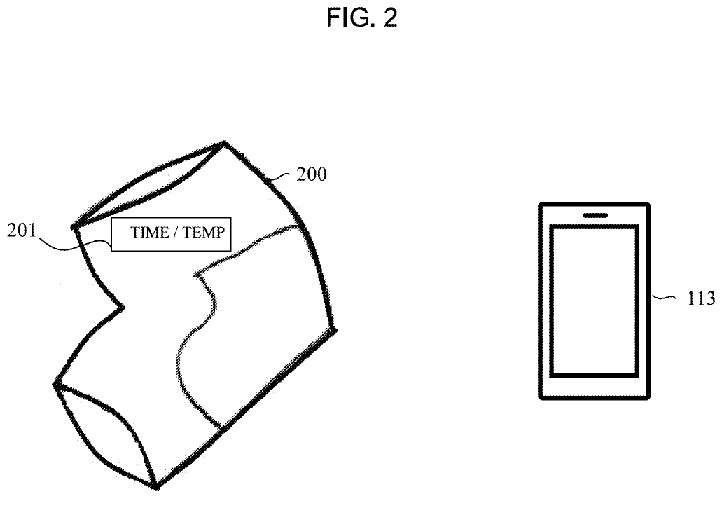

FIG. 2 is an illustration of a wearable device 200 for a knee or an elbow and the mobile device 113 of FIG. 1F. The mobile device 113 is shown in FIG. 2 for convenience. In an alternate embodiment, the wearable device 200 may communicate with any other communication device (e.g., the laptop computer 115, the server 119, and the computers 121 and 123 directly or via the network 117). The wearable device 200 includes all of the components of the wearable device 100 of FIG. 1F but in a wearable form suitable for either a knee or an elbow. The size of the wearable device 200 is different for wearing on a knee and wearing on an elbow due to the differences in size of a knee and an elbow.

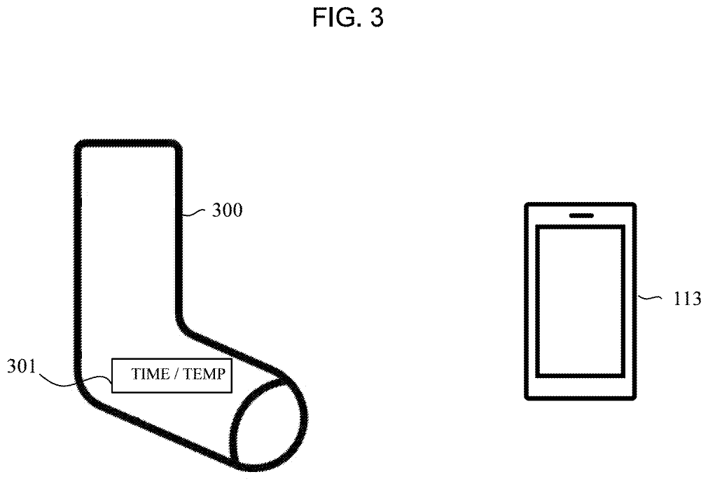

Some embodiments of the housing of the wearable device may include an elastomeric sleeve or other wearable mounting platform (like with laces, Velcro™, or straps) configured to place an active surface of the cooling units (which may also be heating units) adjacent the skin of a human wearing the device. Examples include the sleeves described in the documents incorporated by reference herein, such as a sleeve configured to fit over a user's joint, such as a knee or elbow. Some embodiments may include a plurality of cooling/heating units arranged in an array around a strap or sleeve (or other article of clothing, like a hat, gloves, shoes, a vest, a coat, shorts, underwear, pants, socks, wristbands, headbands, waistbands). In some embodiments, the wearable device including the cooling and heating elements may be integrated into a larger clothing item (e.g., a jacket, shoes, pants, shirt, vest, coat). In some embodiments, each cooling unit may be independently controllable via a microcontroller of the apparatus, and in some cases, sub-regions of the cooling unit may be independently addressable to vary cooling or heating. Some embodiments may include a plurality of such cooling units, like two, four, six, eight, or more. Similarly, the wearable devices depicted in FIGS. 3-8 are contemplated to be constructed of the described elastomeric sleeve or other suitable material and configurations as described.

Referring to FIG. 2, the wearable device 200 includes a display 201, which is equivalent to the display 109 illustrated in FIG. 1F. In some embodiments, the wearable device 200 operates identically to the wearable device 100 described above.

FIG. 3 is an illustration of a wearable device 300 for a foot, ankle, or calf and the mobile device 113 of FIG. 1F. The mobile device 113 is shown in FIG. 3 for convenience. In an alternate embodiment, the wearable device 300 may communicate with any other communication device (e.g., the laptop computer 115, the server 119, and the computers 121 and 123 directly or via the network 117). The wearable device 300 includes all of the components of the wearable device 100 of FIG. 1F but in a wearable form suitable for a foot, ankle, and/or calf.

Referring to FIG. 3, the wearable device 300 includes a display 301, which in some embodiments may be equivalent to the display 109 illustrated in FIG. 1F. The wearable device 300 in some embodiments operates identically to the wearable device 100 described above.

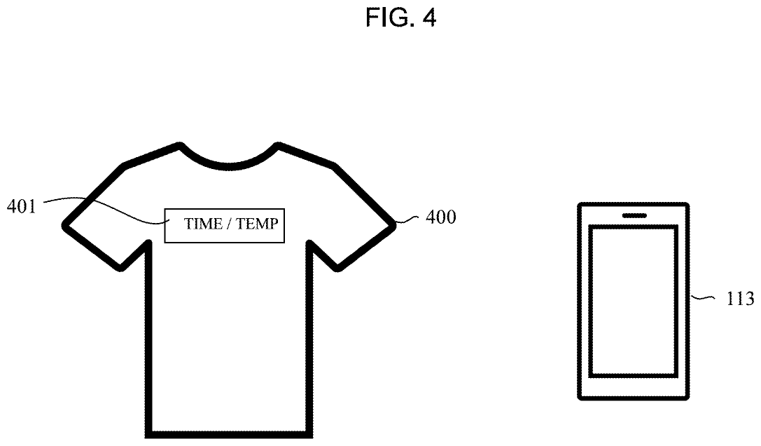

FIG. 4 is an illustration of a wearable device 400 for a chest or a back and the mobile device 113 of FIG. 1F. The mobile device 113 is shown in FIG. 4 for convenience. In an alternate embodiment, the wearable device 400 may communicate with any other communication device (e.g., the laptop computer 115, the server 119, and the computers 121 and 123 directly or via the network 117). The wearable device 400 includes all of the components of the wearable device 100 of FIG. 1F but in a wearable form suitable for wearing on a torso.

Referring to FIG. 4, the wearable device 400 includes a display 401, which in some embodiments may be equivalent to the display 109 illustrated in FIG. 1F. The wearable device 400 in some embodiments operates identically to the wearable device 100 described above.

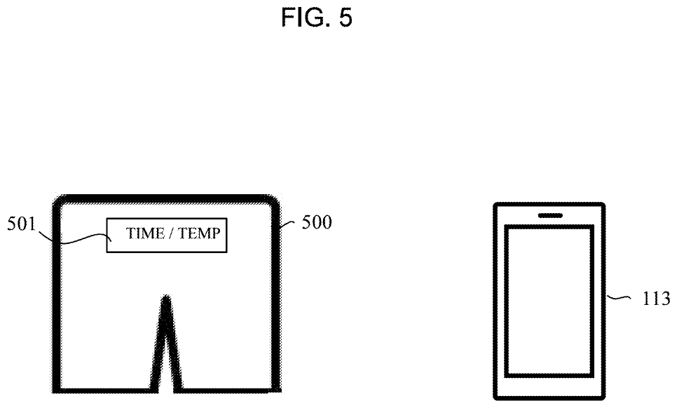

FIG. 5 is an illustration of a wearable device 500 for a waist, groin, glutes, or hamstrings and the mobile device 113 of FIG. 1F. The mobile device 113 is shown in FIG. 5 for convenience. In an alternate embodiment, the wearable device 500 may communicate with any other communication device (e.g., the laptop computer 115, the server 119, and the computers 121 and 123 directly or via the network 117). The wearable device 500 in some embodiments may include all of the components of the wearable device 100 of FIG. 1F but in a wearable form suitable for wearing around the waist and groin.

Referring to FIG. 5, the wearable device 500 includes a display 501, which in some embodiments may be equivalent to the display 109 illustrated in FIG. 1F. The wearable device 500 in some embodiments operates identically to the wearable device 100 described above.

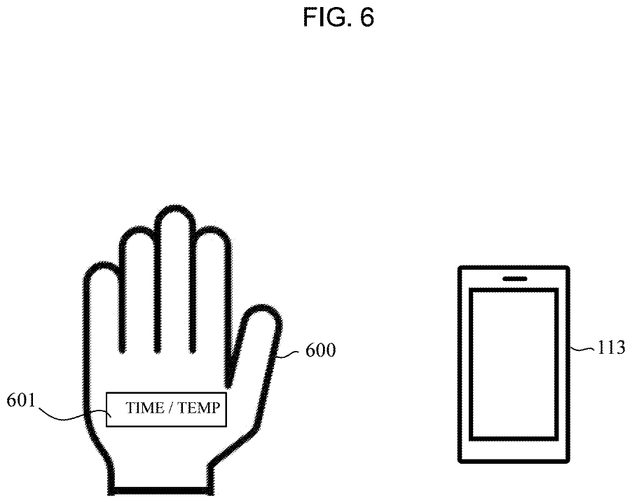

FIG. 6 is an illustration of a wearable device 600 for a hand, finger, knuckle, or wrist and the mobile device 113 of FIG. 1F. The mobile device 113 is shown in FIG. 6 for convenience. In an alternate embodiment, the wearable device 600 may communicate with any other communication device (e.g., the laptop computer 115, the server 119, and the computers 121 and 123 directly or via the network 117). The wearable device 600 includes all of the components of the wearable device 100 of FIG. 1F but in a wearable form suitable for a hand or portions of the hand.

Referring to FIG. 6, the wearable device 600 includes a display 601, which in some embodiments may be equivalent to the display 109 illustrated in FIG. 1F. The wearable device 600 in some embodiments operates identically to the wearable device 100 described above.

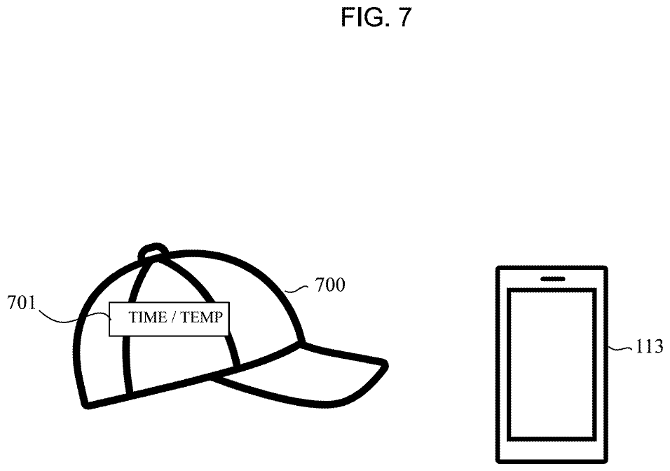

FIG. 7 is an illustration of a wearable device 700 for a head or a forehead and the mobile device 113 of FIG. 1F. The mobile device 113 is shown in FIG. 7 for convenience. In an alternate embodiment, the wearable device 700 may communicate with any other communication device (e.g., the laptop computer 115, the server 119, and the computer 121 and 123 directly or via the network 117). The wearable device 700 includes all of the components of the wearable device 100 of FIG. 1F but in a wearable form suitable for a head and forehead.

Referring to FIG. 7, the wearable device 700 includes a display 701, which in some embodiments may be equivalent to the display 109 illustrated in FIG. 1F. The wearable device 700 in some embodiments operates identically to the wearable device 100 described above.

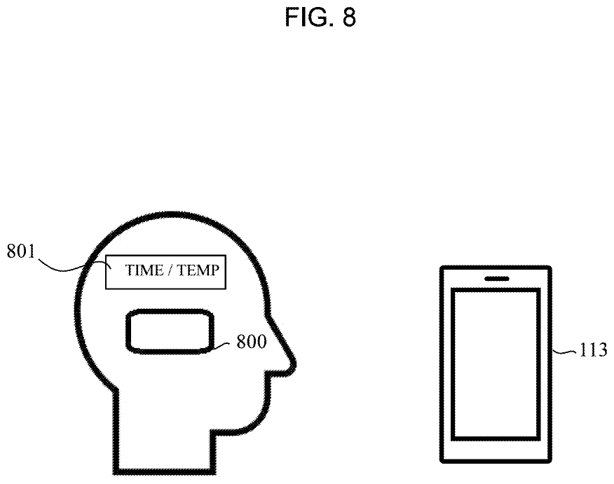

FIG. 8 is an illustration of a wearable device 800 for an eye, a nose, an ear, or a mouth and the mobile device 113 of FIG. 1F. The mobile device 113 is shown in FIG. 8 for convenience. In an alternate embodiment, the wearable device 800 may communicate with any other communication device (e.g., the laptop computer 115, the server 119, and the computers 121 and 123 directly or via the network 117). The wearable device 800 includes all of the components of the wearable device 100 of FIG. 1F but in a wearable form suitable for eyes, ears, nose, and mouth.

Referring to FIG. 8, the wearable device 800 includes a display 801, which is equivalent to the display 109 illustrated in FIG. 1F. The wearable device 800 operates identically to the wearable device 100 described above. The wearable device 800 may be used for a variety of purposes including health, wellness, sleep, and beauty by users targeting inflammation and/or cosmetic surgery recovery needs. However, the present inventive concept is not limited thereto.

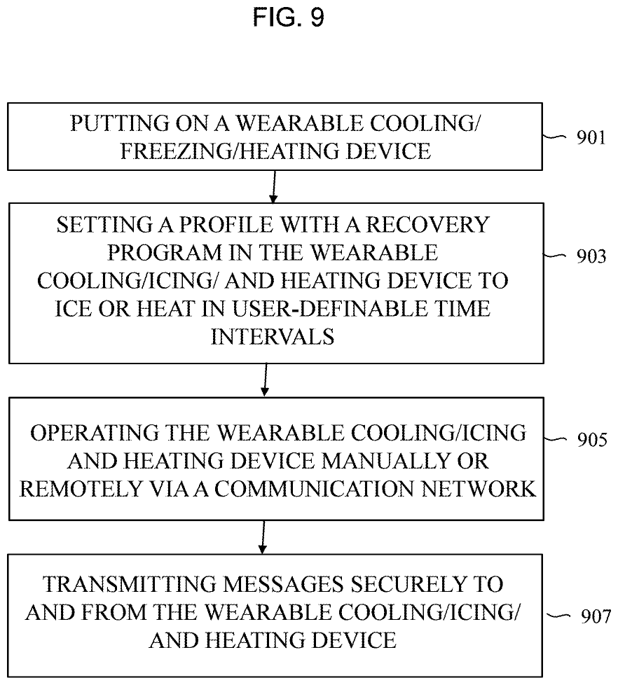

FIG. 9 illustrates a flowchart of a method of icing and heating using a wearable device in accordance with some embodiments.

Referring to FIG. 9, the method of icing and heating includes a first step 901 of putting on a wearable icing and heating device (e.g., wearable device 100) on a body part. For example, a user (e.g., an athlete) has a knee injury. The user will wrap, slide on, or otherwise affix the wearable device to his knee.

The second step 903 of the method is setting a profile with a recovery program in the wearable icing and heating device to cause the wearable icing and heating device to ice or heat in user-definable time intervals. In some embodiments, the timing of the icing and heating of the device is separated in equal time intervals. In some embodiments, the timing of the icing of the device is longer or shorter than the timing of the heating. In some embodiments, the recovery program only includes icing the wearable device. In some embodiments, the recovery program only includes heating the wearable device. It is understood that the recovery program referred to herein describes a single “session” that may include one or more of a heating and cooling of the wearable device for a duration (e.g., 10 minutes). Multiple sessions of the recovery program may be performed by the wearable device. Additionally, a single session (e.g., a recovery program) may be performed by two or more wearable devices. For example, a user places a wearable device on his right and left knee. Each wearable device can be configured to perform the same recovery program at the same time, or individual recovery programs at distinct times. Additionally, sensor data obtained from each wearable device may be used to perform real-time updates to the recovery program.

As an example, the user is wearing a wearable device on his left knee and right knee. The wearable of the left knee senses that the knee is responding well to the heating treatment and communicates that information to the wearable device on the right knee (e.g., via network 117). The wearable device on the right knee may then update its recovery program to increase a temperature of the wearable device or a duration of the recovery program in the heating mode.

The third step 905 of the method is operating the wearable icing and heating device manually or remotely via a communication network (e.g., network 117). In some embodiments, the user (e.g., wearer) may manually select a recovery program. In some embodiments, the wearable device may be remotely accessed (e.g., via mobile device 113). For example, the athlete's trainer may remotely control the recovery program through an integrated application stored on the trainer's cell phone.

The fourth step 907 of the method is transmitting messages securely to and from the wearable icing and heating device. The messages may be information to or from the user or instructions for operating the wearable icing and heating device.

In alternative embodiments, the wearable device includes a feedback loop between a controller, the icing elements and heating elements, and various sensors for safety of the user.

In alternative embodiments, the various sensors may include a heart rate sensor, a pulse oximeter sensor, and various other biometric sensors. The wearable device may further include an epidural layer sensor including a pressure sensor that is configured to function as a predictive tool to estimate a user's expected recovery time. The epidural layer sensor may further be configured to predict a user's susceptibility to re-injury based on various sensors and/or user inputted information including a user diet, amount or quality of sleep, stress, range of motion, height, age, weight, BMI, or the like.

In alternative embodiments, the wearable device may be configured to prevent or reduce a user from experiencing heat exhaustion.

In alternative embodiments, the wearable device may be formed in various shapes and sizes, including a rectangular wrap. However, the present general inventive concept is not limited thereto.

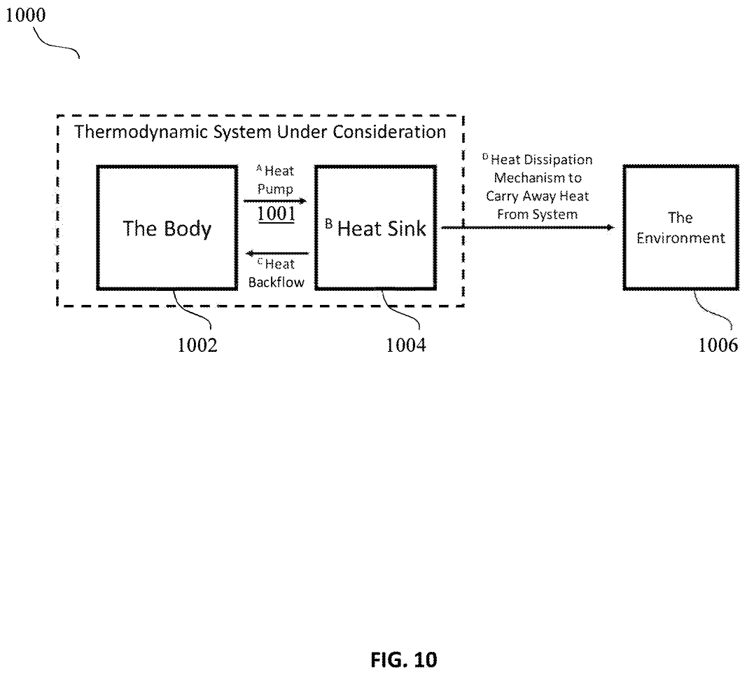

FIG. 10 is a diagram of thermodynamic energy balance applied to the wearable device in accordance with some embodiments. To cool the body (e.g., of a human user wearing a device having the described apparatus), a heat pump 1001 may be used to transport thermal energy from the body 1002 to the heat sink 1004. Buildup of a thermal gradient is undesirable, given that backflow of heat will decrease the efficiency of the device. Therefore, it is beneficial in some cases to eject thermal energy from the immediate system under consideration to the environment 1006. Some embodiments may enhance the dissipation of thermal energy relative to fully passive systems or systems without a phase change of a working fluid.

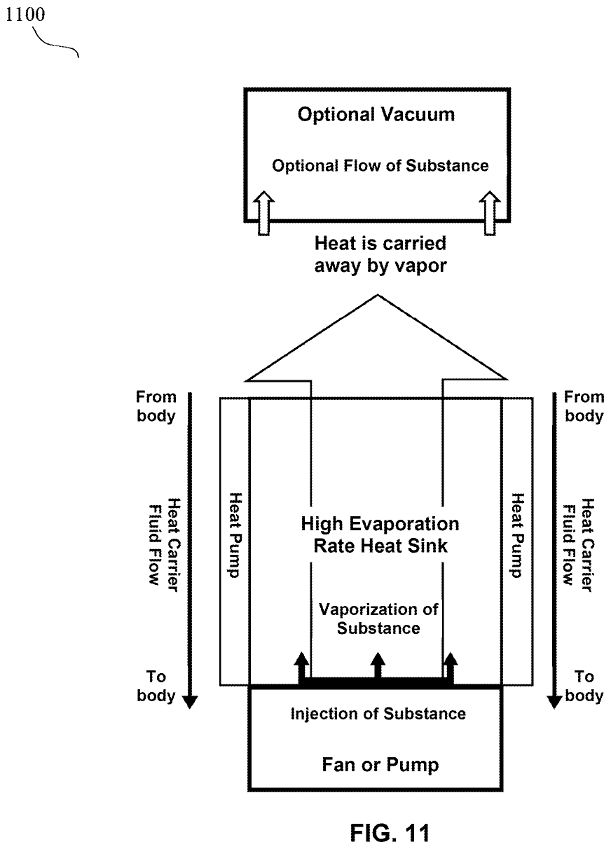

FIG. 11 is a schematic of a rapid heat dissipation for wearable devices in accordance with some embodiments. As shown in rapid heat dissipation system 1100, the substance to be vaporized may be added to the heat sink (e.g., 122), where it is vaporized. The vapors are carried away by advection provided by a fan or a pump (e.g., 130), which may also provide some convective cooling. This lowers the temperature on the side of the heat pump that heat is being transferred to (from the user's body adjacent the other side), thus reducing backflow of heat and increasing efficiency along with coefficient of performance.

In some embodiments, each cooling unit may include the components described above with reference to FIG. 11. Some embodiments may include a blower (e.g., 130) or fan operative to drive ambient air through an airflow path (e.g., 140) along subsequently described components to remove heat from the cooling unit (or supply heat when operating in heating mode to heat, rather than cool, the user's skin). Or some embodiments may use convection or other passive forms of eliciting airflow. In some embodiments, the airflow path may be defined by channels extending between, and in some cases, containing, the described components.

Some embodiments may, at a downstream position of the airflow path (e.g., 140), include a desiccant-based dehumidifier operative to expose the airflow to a surface containing a desiccant (e.g., 132). Various types of desiccants may be used, including liquid or solid desiccants. Examples include Lithium sulfate, sodium sulfate, aluminum sulfate, magnesium sulfate, calcium sulfate, Molecular sieves, Aluminum oxide, Lithium hydroxide, sodium hydroxide, potassium hydroxide, magnesium hydroxide, calcium hydroxide, Lithium oxides, sodium oxide, potassium oxide, magnesium oxide, calcium oxide, Ceramic desiccants, Aluminosilicates, Silicates, Aluminates, Lithium carbonate, sodium carbonate, magnesium carbonate, calcium carbonate, potassium carbonate, Zeolites, Porous carbons, activated charcoal, carbon black. Examples include aqueous solution of organic solvent such as triethylene glycol, diethylene glycol and ethylene glycol, and inorganic aqueous salt solutions such as calcium chloride, lithium chloride, lithium bromide, and calcium bromide; inorganic salt solutions including calcium chloride, lithium chloride, lithium bromide and potassium formate; halide salt solution, like Triethylene glycol, Lithium chloride (LiCl), Calcium chloride (CaCl2) and Lithium bromide (LiBr); Calcium chloride (CaCl2) solution; and Lithium bromide (LiBr) solution. In some cases, solid desiccant beads or coatings, like sodium polyacrylate, zeolite and activated alumina composites, or silica gel (or thermal silica film) may be used as the desiccant.

The desiccant, or in some embodiments, a device having a desiccant, may be static or dynamic. Some embodiments may include a moving plate, like a rotating plate with a solid desiccant coated thereon, in the airflow path (like a series of such plates in spaced relation). Some embodiments may include a regenerator configured to regenerate desiccant after it has absorbed moisture from the ambient airflow. In some embodiments, the regenerator may harvest heat from the below-described thermoelectric heat pumps to raise the temperature of the desiccant to regenerate the desiccant by evaporating off captured moisture. In some cases, the desiccant may be a static desiccant coated on a heat exchanger or impregnated in thermally conductive, breathable fabrics, in the manner described below. A fabric is said to have high thermal conductivity if it has an in-plane thermal conductivity of greater than 0.5 W m-1 K-1 in the direction aligned with the weft yarns.

Some embodiments may include an evaporative cooler downstream in the airflow path from the desiccant-based dehumidifier. In some embodiments, the evaporative cooler may be configured to use water as a working fluid or any of the various other working fluids described above. In some embodiments, the evaporative cooler may include a reservoir of the working fluid, tubing (such as microfluidic channels) or connecting the reservoir to the region of the evaporative cooler where the working fluid is evaporated and a pump or wicking material therebetween to dispense the working fluid in the region where evaporating occurs in the airflow path. Some embodiments may include an ultrasonic transducer disposed in the region where evaporating occurs, in the path of flow of the working fluid into that region. For example, some embodiments may generate a mist of water with the ultrasonic transducer, spray nozzle, atomizer, or the like to facilitate a relatively fast heat exchange with the air, fast evaporation, and a substantial drop in temperature of the air. In some embodiments, the evaporation of the water in the dehydrated air may result in a temperature drop of the airflow into the airflow path.

Some embodiments may then direct the cooled airflow past to a heat exchanger, like an aluminum or copper heat sink with an array of fins, in thermal communication via thermal paste with a heated side of a thermoelectric heat pump, like those described above. Some embodiments may include heat pipes between these components to facilitate heat transfer over a larger distance. In some embodiments, the air cooled by evaporation may be relatively effective to remove thermal energy from the heatsink in virtue of a larger thermal gradient therebetween induced by evaporation of the working fluid. In some cases, heat from the heatsink may driver further evaporation of the above-described mist.

Some embodiments may include a thermoelectric heat pump (e.g., 118), like those described above in thermal communication with the heatsink, for instance adjacent the heatsink with a thermal paste therebetween on a hot-side of the thermoelectric heat pump (for example during a cooling operation), with a cool-side of the thermoelectric heat pump positioned adjacent the skin of a person or other organism wearing the device having the cooling unit. Some embodiments may include various thermally conductive fabrics, like those described below between the cooling-side (during a cooling operation, as rules may be reversed during heating operation) between the of the heat pump and the user's skin.

In some embodiments, the thermoelectric heat pump may be a flexible thermoelectric heat pump configured to conform to the shape of the user's body, for instance, upon being biased against the user's body by an elastomeric sleeve of the apparatus, for instance, an elastane fabric sleeve. Examples of flexible thermoelectric heat pumps include those available from TEGway, at #202, 56 Sinilseo-ro 67beon-gil, Daedeok-gu, Daejeon, Republic of Korea (34325). In some cases, the flexible thermoelectric heat pump may have an operative surface area of more than 5, 10, or 20 square cm. Or some embodiments may include a rigid thermoelectric key pump. In some cases, the thermoelectric heat pump is a Bismuth Telluride (BiTe) based device. A thermoelectric heat pump (or other sheet of material) is said to be “flexible” if it has an ASTM (American Society for Testing and Materials) D2176 endurance of greater than 100 cycles.

In some embodiments, the airflow path may next pass through an exhaust port through which air carrying heat away from the heat exchanger is exhausted into the ambient environment, along with evaporated working fluid.

Some embodiments may include an evaporative cooler that is passively implemented, in a flexible material, along with a flexible thermoelectric cooler. A flexible stack of such materials is expected to facilitate relatively large contact areas with the skin and relatively low thermal resistance to heat removal from the skin of the user. For example, some embodiments may be implemented in the following stack.

Some embodiments may place adjacent the skin of the user, for instance, by biasing the stack against the user skin with one of the above-describe sleeves, a thermally conductive fabric. Examples including woven ultrahigh molecular weight polyethylene. Other examples include Stretch conductive fabric 4900 from Holland Shielding Systems BV of 3316BP Dordrecht, the Netherlands.

Some embodiments may include a flexible thermoelectric heat pump, like those described above. In some embodiments, the stack may be uniformly arrayed around the entirety of the sleeve (or other type of garment) or at various areas of the sleeve in isolated islands.

Next, in some embodiments, a heat exchanger may be integrated with a desiccant that holds a reservoir of the working fluid. For example, a desiccant-coated heat exchanger may be positioned adjacent the flexible thermoelectric heat pump (on a hot side of the heat pump during a cooling operation). In some embodiments, the heat exchanger is implemented with solid desiccant beads, like silica gel or sodium polyacrylic (which in some cases can 100 to 1000 times its mass in water), sandwiched between layers of a thermally conductive fabric, like a woven thermally conductive fabric that is breathable on at least an external side of the stack. For instance, such beads may be disposed in an array of pockets formed between such layers of fabric by quilting the thermally conductive fabrics together.

Some embodiments may include an elastic fabric, like a breathable elastic fabric at an outer portion of the stack, like in a sleeve configured to bias the slot stack against the skin of the user. In some embodiments, the outer portion may include an array of apertures to further facilitate airflow. Resiliency, used herein interchangeably with elasticity, may be measured with ASTM D4964-96, using the techniques described by Jinjun et al, in a paper titled “The Poisson Ratio and Modulus of Elastic Knitted Fabric,” published in the Textile Research Journal, 1 Vol 80(18): 1965-1969 DOI: 10.1177/0040517510371864, the contents of which are hereby incorporated by reference, with such fabrics exhibiting a uniaxial tensile Young's elastic modulus of less than 50 kPa in at least one direction.

In use of some embodiments of this stack, the user may first load the desiccant with working fluid. For example, the user may place the device having the stack in a bath of the working fluid, for instance, in a sink full of water, or under running water from a sink to load the desiccant with water. Next, the sleeve may be placed on the user's body. In some embodiments, upon placing the sleeve on the user's body, the external elastomeric layer of the stack may bias the stack against the user's body, and the various layers of the stack may can formally conform to the shape of the user's body as they flex. Next, in some embodiments, electrical energy drawn from batteries integrated the sleeve may drive a current through the thermoelectric heat pump, causing the heat pump to cool on one side (while absorbing heat from the user's body, cooling the body) adjacent the user's body and causing the heat pump to warm on an external side. In some embodiments, the elevated temperature on the external side of the thermoelectric heat pump may cause heat energy to flow through the external thermally conductive fabric, or into the working-fluid impregnated desiccant (or other absorbent material, like porous carbon, activated charcoal, paper towel, cotton, rayon, synthetic fibers, glass wool, inorganic fibers, bio-derived fibers, porous pellets, sponges, etc.), which may regenerate the desiccant, causing the water therein to evaporate, and thereby carrying heat energy away from the apparatus. The evaporating working fluid may flow through apertures in the external elastomeric layer of the stack, carrying heat energy away from the device. In some cases, the stack may operate without pumping the working fluid, by using the desiccant to hold the working fluid adjacent the thermoelectric heat pump. Contemplated absorbent materials also include the following: Hydrogel and hydrogel absorbent, Titanium dioxide, Zinc oxide, Mixed metal oxides, Boron oxides, Synthetic resins, Cellulose, Lignin, Chromatography packing media, Silicones, siloxanes, modified silicon-based polymers, Aluminates, titanates, Alums and related compounds, Borax, borates, boronic acids, Dendrimers, hairbrush polymers, and polymer networks, Covalent organic frameworks, metal-organic frameworks, ZIFs, network solids, porous network solids, Zeolites, Microporous polymers and reticulated polymers, Reticulated Carbons, Carbon fibers, Surface-modified carbons, Graphene oxide, Various 2D and 1D materials, Various porous media Emulsions, Multi-phase fluids, Micelles, Naturally derived or inspired substances, Proteins, peptides, peptoids, carbohydrates, and chemically modified carbohydrates. Also, Use of various surfactants to promote wetting high surface area porous solids or to make phases compatible. In some cases, cooling operations may be initiated responsive to detecting sweat on the user's skin, for instance, with a galvanic sensor, to amplify the user's natural response.

In some embodiments, a working fluid, like a liquid, may be drawn over (e.g., adjacent, in thermal contact) the heat sink with a vacuum (e.g., 134) instead of, or in addition to, with a pump. In some embodiments, the vacuum approach is expected to impose less challenging sealing design challenges when protecting against undesirable leaks of the working fluid. In some cases, the vacuum may be implemented with a micro diaphragm vacuum pump or other miniature vacuum pump, like a miniature linear vacuum pump, a miniature rotary vane vacuum pump, or a miniature Wob-L piston vacuum pump. In some cases, a working fluid may be poured onto the heat sink and drawn away into one of a pair of flexible bladders by one or more vacuum pumps. In some cases, the vacuum pump may be used to implement a closed refrigeration cycle to draw heat away from the heat exchanger.

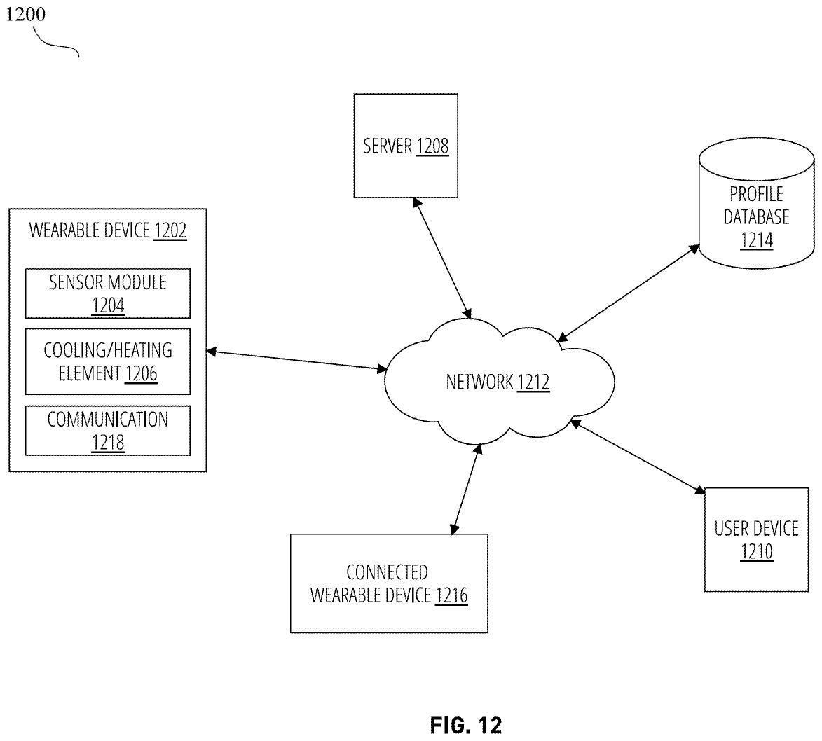

FIG. 12 illustrates a block diagram of a representative system including one or more connected wearable devices in accordance with some embodiments.

The computing system 1200 may include a wearable device 1202, connected wearable device 1216, server 1208, user device 1210 (e.g., client device 113, FIG. 1F), and profile database 1214. The wearable device (e.g., wearable device 1202 or 1216) may include a sensor module 1204, a cooling/heating element 1206 (e.g., cooling/icing/heating element 112, FIG. 1F), and communication module 1218.

Wearable device 1202 is an example of a wearable device that includes a sensor module 1204 and a cooling/heating element 1206. Additional modules may be included but are not illustrated in FIG. 12. Wearable device 1202 may be modularly configurable, meaning, one or more modules may be added or removed from the wearable device 1202 1202. Wearable device 1216 may be substantially the same device as wearable device 1202.

Sensor module 1204 of wearable device 1202 may include a plurality of biometric sensors including heart rate sensor, pulse oximeter sensor, temperature sensors, pressure sensors, cameras, and other various configurable sensors. Each of the sensors may be configured to provide sensor data to an onboard computer system or transmitted via network 1212 to one or more computing devices including server 1208 and/or user device 1210.

The communication module 1224 may be used by the wearable device 1216 to communicate with another wearable device (e.g., wearable device 1202), server 1208, user device 1210, profile database 1214, or any other networked peripheral device.

Profile database 1214 may include a directory of profiles corresponding to users of the wearable device and providers of the wearable device. For example, profile database 1214 includes historical usage information, average therapy session durations, temperature data, error frequencies, and other metadata collected by the wearable devices. Additional information may be stored at the profile database including optimization algorithms, one or more machine learning models, training data, historical data, metadata, and performance data.

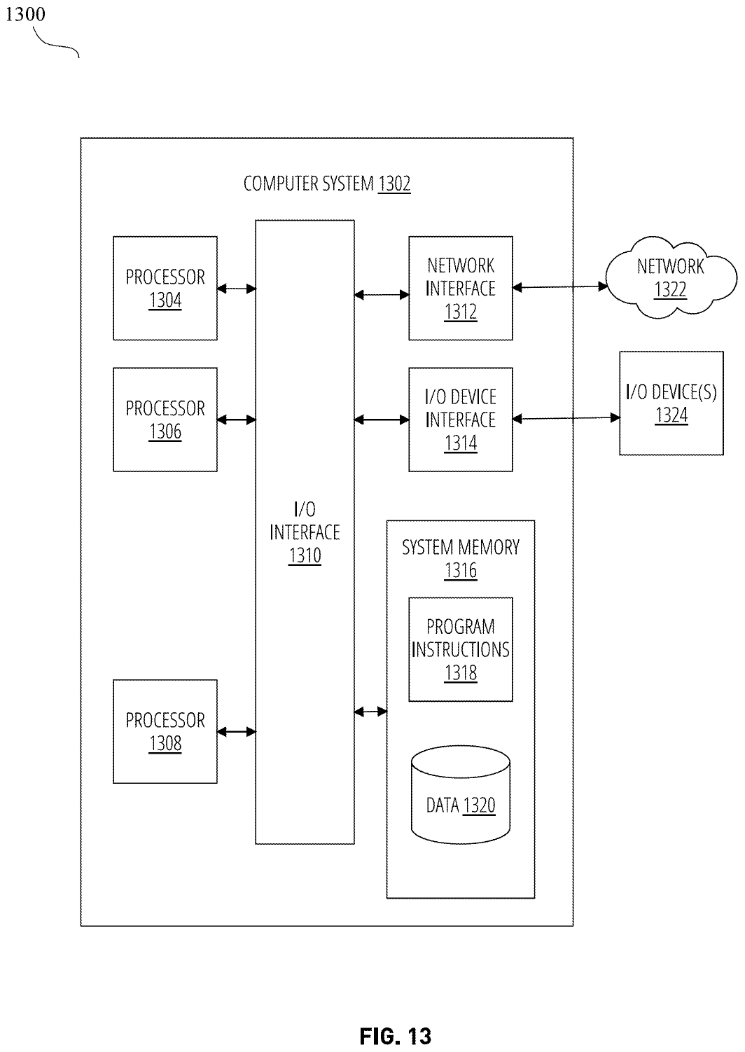

FIG. 13 illustrates an example computing device by which the above techniques may be implemented.

Various portions of systems and methods described herein, may include, or be executed on one or more computer systems similar to computing system 1300. Further, processes and modules described herein may be executed by one or more processing systems similar to that of computing system 1300.

Computer system 1302 may include one or more processors (e.g., processors 1304, 1306, 13013) coupled to system memory 1316, an input/output 1314, and a network interface 1312 via an input/output I/O interface 1310. A processor may include a single processor or a plurality of processors (e.g., distributed processors). A processor may be any suitable processor capable of executing or otherwise performing instructions. A processor may include a central processing unit (CPU) that conducts program instructions to perform the arithmetical, logical, and input/output operations of computer system 1302. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (e.g., system memory 1316). Computer system 1302 may be a units-processor system including one processor (e.g., processor 1304), or a multi-processor system including any number of suitable processors (e.g., 1304-1308). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus can also be implemented as, special purpose logic circuitry, e.g., an FPGA (field programmable gate array) or an ASIC (application specific integrated circuit). Computer system 1302 may include a plurality of computing devices (e.g., distributed computer systems) to implement various processing functions.

I/O device interface 1314 may provide an interface for connection of one or more I/O devices 1324 to computer system 1302. I/O devices may include devices that receive input (e.g., from a user) or output information (e.g., to a user). I/O devices 1324 may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. I/O devices 1324 may be connected to computer system 1302 through a wired or wireless connection. I/O devices 1324 may be connected to computer system 1302 from a remote location. I/O device(s) 1324 located on remote computer system, for example, may be connected to computer system 1302 via a network 1322 and network interface 1312.

Network interface 1312 may include a network adapter that provides for connection of computer system 1302 to a network 1322. Network interface 1312 may facilitate data exchange between computer system 1302 and other devices connected to the network. Network interface 1312 may support wired or wireless communication. The network may include an electronic communication network, such as the Internet, a local area network (LAN), a wide area network (WAN), a cellular communications network, or the like.

System memory 1316 may be configured to store program instructions 1308 or data 1320. Program instructions 1318 may be executable by a processor (e.g., one or more of Processors 1304-1308) to implement one or more embodiments of the present techniques. Instructions 1318 may include modules of computer program instructions for implementing one or more techniques described herein with regard to various processing modules. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

System memory 1316 may include a tangible program carrier having program instructions stored thereon. A tangible program carrier may include a non-transitory computer readable storage medium. A non-transitory computer readable storage medium may include a machine-readable storage device, a machine-readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random-access memory (SRAM), synchronous dynamic RAM (SDRAM)), bulk storage memory (e.g., CD-ROM and/or DVD-ROM, hard drives), or the like. System memory 1316 may include a non-transitory computer readable storage medium that may have program instructions stored thereon that are executable by a computer processor (e.g., one or more of processor 1304-13013) to cause the subject matter and the functional operations described herein. A memory (e.g., system memory 1316) may include a single memory device and/or a plurality of memory devices (e.g., distributed memory devices).

I/O interface 1310 may be configured to coordinate I/O traffic between processor 1304-13013, system memory 1316, network interface 1312, I/O devices 1324, and/or other peripheral devices. I/O interface 1310 may perform protocol, timing, or other data transformations to convert data signals from one component (e.g., System memory 1316) into a format suitable for use by another component (e.g., processors 1304-13013). I/O Interface 1310 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computer system 1302 or multiple computing systems 1300 configured to host different portions or instances of embodiments. Multiple computing systems 1300 may provide for parallel or sequential processing/execution of one or more portions of the techniques described herein.

Those skilled in the art will appreciate that computing system 1300 is merely illustrative and is not intended to limit the scope of the techniques described herein. Computing system 1300 may include any combination of devices or software that may perform or otherwise provide for the performance of the techniques described herein. For example, computing system 1300 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, or a Global Positioning System (GPS), or the like. Computing system 1300 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all the software components may execute in memory on another device and communicate with the illustrated computer system via inter-computer communication. Some or all the system components or data structures may also be stored (e.g., as instructions or structured data) on a computer-accessible medium or a portable article to be read by an appropriate drive, various examples of which are described above. In some embodiments, instructions stored on a computer-accessible medium separate from computing system 1300 may be transmitted to computing system 1300 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present disclosure may be practiced with other computer system configurations.

In block diagrams, illustrated components are depicted as discrete functional blocks, but embodiments are not limited to systems in which the functionality described herein is organized as illustrated. The functionality provided by each of the components may be provided by software or hardware modules that are differently organized than is presently depicted, for example such software or hardware may be intermingled, conjoined, replicated, broken up, distributed (e.g., within a data center or geographically), or otherwise differently organized. The functionality described herein may be provided by one or more processors of one or more computers executing code stored on a tangible, non-transitory, machine readable medium. In some cases, third party content delivery networks may host some or all the information conveyed over networks, in which case, to the extent information (e.g., content) is said to be supplied or otherwise provided, the information may be provided by sending instructions to retrieve that information from a content delivery network.

The reader should appreciate that the present application describes several independently useful techniques. Rather than separating those techniques into multiple isolated patent applications, applicants have grouped these techniques into a single document because their related subject matter lends itself to economies in the application process. But the distinct advantages and aspects of such techniques should not be conflated. In some cases, embodiments address all of the deficiencies noted herein, but it should be understood that the techniques are independently useful, and some embodiments address only a subset of such problems or offer other, unmentioned benefits that will be apparent to those of skill in the art reviewing the present disclosure. Due to costs constraints, some techniques disclosed herein may not be presently claimed and may be claimed in later filings, such as continuation applications or by amending the present claims. Similarly, due to space constraints, neither the Abstract nor the Summary of the Invention sections of the present document should be taken as containing a comprehensive listing of all such techniques or all aspects of such techniques.

It should be understood that the description and the drawings are not intended to limit the present techniques to the particular form disclosed, but to the contrary, the intention is to cover all modifications, equivalents, and alternatives falling within the spirit and scope of the present techniques as defined by the appended claims. Further modifications and alternative embodiments of various aspects of the techniques will be apparent to those skilled in the art in view of this description. Accordingly, this description and the drawings are to be construed as illustrative only and are for the purpose of teaching those skilled in the art the general manner of conducting the present techniques. It is to be understood that the forms of the present techniques shown and described herein are to be taken as examples of embodiments. Elements and materials may be substituted for those illustrated and described herein, parts and processes may be reversed or omitted, and certain features of the present techniques may be utilized independently, all as would be apparent to one skilled in the art after having the benefit of this description of the present techniques. Changes may be made in the elements described herein without departing from the spirit and scope of the present techniques as described in the following claims. Headings used herein are for organizational purposes only and are not meant to be used to limit the scope of the description.

As used throughout this application, the word “may” is used in a permissive sense (i.e., meaning having the potential to), rather than the mandatory sense (i.e., meaning must). The words “include,” “including,” and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, i.e., encompassing both “and” and “or.” Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Similarly, reference to “a computer system” performing step A and “the computer system” performing step B can include the same computing device within the computer system performing both steps or different computing devices within the computer system performing steps A and B. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X'ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device. Features described with reference to geometric constructs, like “parallel,” “perpendicular/orthogonal,” “square,” “cylindrical,” and the like, should be construed as encompassing items that substantially embody the properties of the geometric construct, e.g., reference to “parallel” surfaces encompasses substantially parallel surfaces. The permitted range of deviation from Platonic ideals of these geometric constructs is to be determined with reference to ranges in the specification, and where such ranges are not stated, with reference to industry norms in the field of use, and where such ranges are not defined, with reference to industry norms in the field of manufacturing of the designated feature, and where such ranges are not defined, features substantially embodying a geometric construct should be construed to include those features within 15% of the defining attributes of that geometric construct. The terms “first”, “second”, “third,” “given” and so on, if used in the claims, are used to distinguish, or otherwise identify, and not to show a sequential or numerical limitation. As is the case in ordinary usage in the field, data structures and formats described with reference to uses salient to a human need not be presented in a human-intelligible format to constitute the described data structure or format, e.g., text need not be rendered or even encoded in Unicode or ASCII to constitute text; images, maps, and data-visualizations need not be displayed or decoded to constitute images, maps, and data-visualizations, respectively; speech, music, and other audio need not be emitted through a speaker or decoded to constitute speech, music, or other audio, respectively. Computer implemented instructions, commands, and the like are not limited to executable code and can be implemented in the form of data that causes functionality to be invoked, e.g., in the form of arguments of a function or API call. To the extent bespoke noun phrases (and other coined terms) are used in the claims and lack a self-evident construction, the definition of such phrases may be recited in the claim itself, in which case, the use of such bespoke noun phrases should not be taken as invitation to impart additional limitations by looking to the specification or extrinsic evidence.

In this patent, to the extent any U.S. patents, U.S. patent applications, or other materials (e.g., articles) have been incorporated by reference, the text of such materials is only incorporated by reference to the extent that no conflict exists between such material and the statements and drawings set forth herein. In the event of such conflict, the text of the present document governs, and terms in this document should not be given a narrower reading in virtue of the way in which those terms are used in other materials incorporated by reference.

The present techniques will be better understood with reference to the following enumerated embodiments:

1. An apparatus, comprising a strap or sleeve operative to attach to a user's body; a thermoelectric heat pump coupled to the strap or sleeve at a position configured to place a first surface of the thermoelectric heat pump adjacent the user's body; a battery configured to supply power to the thermoelectric heat pump; a driver circuit in series between the battery and the thermoelectric heat pump, the driver circuit being operative to driver electrical power to the thermoelectric heat pump responsive to control signals; a microcontroller configured to provide the control signals to control the thermoelectric heat pump responsive to code executed by the microcontroller and stored in a tangible, machine-readable medium accessible to the microcontroller; and an evaporative heat exchanger thermally coupled to a second surface of the thermoelectric heat pump, the evaporative heat exchanger being configured to accept thermal energy from the second surface of the thermoelectric heat pump, conduct the thermal energy to a working fluid in a liquid state, evaporate the working fluid, and exhaust the evaporated working fluid into an ambient environment to thereby expel at least some of the thermal energy.
2. The apparatus of embodiment 1, wherein: the working fluid is water; and the apparatus comprises a fan or blower configured to blow air adjacent or through the evaporative heat exchanger, along an airflow path.
3. The apparatus of embodiment 2, comprising: an ultrasonic transducer in a fluid flow path of the water and operative to generate a mist of the water in the airflow path.
4. The apparatus of embodiment 2, comprising: a desiccant-based dehumidifier in the airflow path upstream from a region in the airflow path in which the water evaporates.
5. The apparatus of embodiment 1, wherein: the thermoelectric heat pump is a flexible thermoelectric heat pump having a folding endurance under ASTM (American Society for Testing and Materials) D2176 of greater than 1,000 cycles.
6. The apparatus of embodiment 5, wherein: the evaporative heat exchanger is a flexible evaporative heat exchanger having a folding endurance under ASTM D2176 of greater than 1,000 cycles.
7. The apparatus of embodiment 6, wherein: the flexible evaporative heat exchanger comprises a desiccant configured to absorb the working fluid before cooling operations and release the working fluid during operations.
8. The apparatus of embodiment 7, wherein the flexible evaporative heat exchanger comprises a thermally conductive fabric containing the desiccant.
9. The apparatus of embodiment 1, wherein the evaporative heat exchanger comprises a desiccant-coated heat sink or absorbent material positioned adjacent the heat sink.
10. A heat dissipation system for wearable devices that uses vaporization of a harmless substance combined with advection.
11. The substance according to embodiment 10 may include any or a combination of the following compounds or mixtures: water, propylene glycol, glycerin, or any other harmless substance that has a significantly large latent heat of vaporization greater than 15000 J/mol or 300 J/g.
12. The system according to embodiment 10, wherein the vaporization absorbs heat that is transported away from a user's body and/or heat that is generated by operation of electronic components including but not limited to Peltier modules, electronic hardware, electronic chips, controllers, circuit boards, and other compound electronic devices.
13. The system according to embodiment 10 and 13, wherein the vapor is exhausted into the surrounding environment through the use of advection, thereby carrying away heat.
14. A wearable device, comprising: a housing configured to encompass at least a portion of a body part of a wearer; a plurality of biometric sensors coupled to the housing; a controller communicably coupled to the housing and the plurality of biometric sensors; a communication channel for communicating with an external communication device; a temperature regulator coupled to the controller and housing, the temperature regulator being configured to controllably adjust a temperature of the housing; and a power unit configured to provide power to the temperature regulator.
15. The wearable device of embodiment 14, wherein the temperature regulator is configured to fluctuate between causing the housing to be at a first temperature and a second temperature that is hotter than the first temperature.
16. The wearable device of embodiment 14, wherein the device is configured to: receive biometric sensor information from one or more of the plurality of biometric sensors; provide the biometric sensor information via communication channel; receive one or more commands from the external communication device including a target temperature in response to the biometric sensor information; determine a current temperature of the temperature regulator is not of a same temperature as the target temperature; and cause the controller to update the current temperature of the temperature regulator to be at the target temperature, wherein the one or more commands includes a target temperature range and a timing interval.
17. The wearable device of embodiment 14, comprising: a display coupled to the housing configured to display a current temperature of the temperature regulator, wherein the display is configured to receive a manual input of a desired temperature and a desired timing interval.
18. The wearable device of embodiment 14, wherein the communication channel is configured to receive one or more instructions via a wireless communication to control the housing through a connected application, and wherein the controller is configured to communicate with a secondary wearable device.
19. The wearable device of embodiment 18, wherein the wearable device is configured to communicate a first temperature and timing interval to the secondary wearable device.
20. The wearable device of embodiment 14, wherein the housing is configured for encompassing at least a user's knee, elbow, foot, chest, back, torso, waist, groin, hand, head, forehead, eye, nose, ear, mouth, shoulder, wrist, or ankle.
21. The wearable device of embodiment 14, wherein the temperature regulator is a thermoelectric heat pump coupled to the housing and configured to place a first surface of the thermoelectric heat pump adjacent the body part.
22. The wearable device of embodiment 21, comprising: an evaporative heat exchanger thermally coupled to a second surface of the thermoelectric heat pump, the evaporative heat exchanger being configured to accept thermal energy from the second surface of the thermoelectric heat pump, conduct the thermal energy to a working fluid in a liquid state, evaporate the working fluid, and exhaust the evaporated working fluid into an ambient environment.
23. The wearable device of embodiment 14, comprising: a fan configured to blow air adjacent to or through the evaporative heat exchanger, along an airflow path; and an ultrasonic transducer configured to generate a mist of the working fluid in the airflow path.
24. The wearable device of embodiment 22, comprising a vacuum pump configured to remove the working fluid from either the first surface or the second surface of the thermoelectric heat pump.

What is claimed is:

1. An apparatus, comprising:
a strap or sleeve operative to attach to a user's body;
a thermoelectric heat pump coupled to the strap or sleeve at a position configured to place a first surface of the thermoelectric heat pump adjacent the user's body;
a battery configured to supply power to the thermoelectric heat pump;
a driver circuit in series between the battery and the thermoelectric heat pump, the driver circuit being operative to driver electrical power to the thermoelectric heat pump responsive to control signals;
a microcontroller configured to provide the control signals to control the thermoelectric heat pump responsive to code executed by the microcontroller and stored in a tangible, machine-readable medium accessible to the microcontroller; and
an evaporative heat exchanger thermally coupled to a second surface of the thermoelectric heat pump, the evaporative heat exchanger being configured to accept thermal energy from the second surface of the thermoelectric heat pump, conduct the thermal energy to a working fluid in a liquid state, evaporate the working fluid, and exhaust the evaporated working fluid into an ambient environment to thereby expel at least some of the thermal energy.

2. The apparatus of claim 1, wherein:
the working fluid is water; and
the apparatus comprises a fan or blower configured to blow air adjacent or through the evaporative heat exchanger, along an airflow path.

3. The apparatus of claim 2, comprising:
an ultrasonic transducer in a fluid flow path of the water and operative to generate a mist of the water in the airflow path.

4. The apparatus of claim 2, comprising:
a desiccant-based dehumidifier in the airflow path upstream from a region in the airflow path in which the water evaporates.

5. The apparatus of claim 1, wherein:
the thermoelectric heat pump is a flexible thermoelectric heat pump having a folding endurance under ASTM (American Society for Testing and Materials) D2176 of greater than 1,000 cycles.

6. The apparatus of claim 5, wherein:
the evaporative heat exchanger is a flexible evaporative heat exchanger having a folding endurance under ASTM D2176 of greater than 1,000 cycles.

7. The apparatus of claim 6, wherein:
the flexible evaporative heat exchanger comprises a desiccant configured to absorb the working fluid before cooling operations and release the working fluid during operations.

8. The apparatus of claim 7, wherein the flexible evaporative heat exchanger comprises a thermally conductive fabric containing the desiccant.

9. The apparatus of claim 1, wherein the evaporative heat exchanger comprises a desiccant-coated heat sink or absorbent material positioned adjacent the heat sink.

10. A wearable device, comprising:
a housing configured to encompass at least a portion of a body part of a wearer;
a plurality of biometric sensors coupled to the housing;
a controller communicably coupled to the housing and the plurality of biometric sensors;
a communication channel for communicating with an external communication device;
a temperature regulator coupled to the controller and housing, the temperature regulator being configured to controllably adjust a temperature of the housing; and
a power unit configured to provide power to the temperature regulator.

11. The wearable device of claim 10, wherein the temperature regulator is configured to fluctuate between causing the housing to be at a first temperature and a second temperature that is hotter than the first temperature.

12. The wearable device of claim 10, wherein the device is configured to:
receive biometric sensor information from one or more of the plurality of biometric sensors;
provide the biometric sensor information via communication channel;
receive one or more commands from the external communication device including a target temperature in response to the biometric sensor information;
determine a current temperature of the temperature regulator is not of a same temperature as the target temperature; and
cause the controller to update the current temperature of the temperature regulator to be at the target temperature, wherein the one or more commands includes a target temperature range and a timing interval.

13. The wearable device of claim 10, comprising: a display coupled to the housing configured to display a current temperature of the temperature regulator, wherein the display is configured to receive a manual input of a desired temperature and a desired timing interval.

14. The wearable device of claim 10, wherein the communication channel is configured to receive one or more instructions via a wireless communication to control the housing through a connected application, and wherein the controller is configured to communicate with a secondary wearable device.

15. The wearable device of claim 14, wherein the wearable device is configured to communicate a first temperature and timing interval to the secondary wearable device.

16. The wearable device of claim 10, wherein the housing is configured for encompassing at least a user's knee, elbow, foot, chest, back, torso, waist, groin, hand, head, forehead, eye, nose, ear, mouth, shoulder, wrist, or ankle.

17. The wearable device of claim 10, wherein the temperature regulator is a thermoelectric heat pump coupled to the housing and configured to place a first surface of the thermoelectric heat pump adjacent the body part.

18. The wearable device of claim 17, comprising:
an evaporative heat exchanger thermally coupled to a second surface of the thermoelectric heat pump, the evaporative heat exchanger being configured to accept thermal energy from the second surface of the thermoelectric heat pump, conduct the thermal energy to a working fluid in a liquid state, evaporate the working fluid, and exhaust the evaporated working fluid into an ambient environment.

19. The wearable device of claim 18, comprising:
a fan configured to blow air adjacent to or through the evaporative heat exchanger, along an airflow path; and
an ultrasonic transducer configured to generate a mist of the working fluid in the airflow path.

20. The wearable device of claim 18, comprising a vacuum pump configured to remove the working fluid from either the first surface or the second surface of the thermoelectric heat pump.

21. The wearable device of claim 10, comprising: means for dissipating thermal energy through vaporization of a substance.

22. The wearable device of claim 10, comprising: means for dissipating thermal energy using at least one of advection, a phase transition, or a combination thereof.
